(19) United States
(12) Patent Application Publication
(10) Pub. No.: US 2024/0408214 A1
(43) Pub. Date: Dec. 12, 2024

(54) NANOPARTICULATE MONOBENZONE FOR TREATING MELANOMA

(71) Applicant: Northwestern University, Evanston, IL (US)

(72) Inventors: Isabelle Caroline Le Poole, Evanston, IL (US); SonBinh T. Nguyen, Evanston, IL (US)

(21) Appl. No.: 18/694,853

(22) PCT Filed: Sep. 26, 2022
(86) PCT No.: PCT/US2022/077022

§ 371 Date: Mar. 22, 2024

Related U.S. Application Data
(60) Provisional application No. 63/261,599, filed on Sep. 24, 2021.

(51) Int. Cl. A61K 47/54; A61K 9/127; A61K 31/05; A61P 35/00

(52) U.S. Cl. CPC A61K 47/542; A61K 9/1271; A61K 9/1277; A61K 31/05; A61P 35/00

(57) ABSTRACT

Disclosed herein is nanoparticulate monobenzone comprising a nanostructured carrier and monobenzone or a conjugate thereof embedded within the nanostructured carrier and methods of making and using the same. The nanostructured carrier can be a liposome o a metal-organic framework. Besides the monobenzone conjugate, the nanostructured carrier may further comprise a detectable label, a stabilizing agent, a targeting agent, linking agent, or any combination thereof and tailored for particular uses. The field of the invention relates to the use of nanoparticulate monobenzone in pharmaceutical compositions for treating melanoma.

### CROSS-REFERENCE TO RELATED APPLICATIONS

The present application claims priority to U.S. Provisional Patent Application No. 63/261,599, filed Sep. 24, 2021, the entire contents of which are hereby incorporated by reference.

### FIELD OF THE INVENTION

The disclosed technology is generally directed to a nanoparticulate composition. More particularly the technology is directed to a nanoparticle composition having a pharmaceutical application.

### BACKGROUND OF THE INVENTION

Melanoma (and malignant melanoma) is a form of skin cancer that develops from melanocytes, the pigment-producing cells. Melanocytes and melanoma cells are exclusively capable of synthesizing melanin pigment inside melanosomes, lysosome-like organelles. The first steps in melanin synthesis are the oxidation of the amino acid L-tyrosine to dopa-quinone, catalyzed by the enzyme tyrosinase and tyrosinase-related protein 1 (TRP1). As melanoma cells express melanosomal enzymes, targeting this melanogenic pathway by analogs of the melanin synthetic pathway provides a unique strategy for developing new treatments for melanoma.

Among the potential drug candidates in this approach are substituted phenols, which are structurally similar to tyrosine and can be converted to a product that is toxic to the host cells. As a model phenolic drug, monobenzyl ether of hydroquinone (MBEH), aka monobenzone, can indeed reduce melanoma growth when applied topically. However, melanomas rapidly metastasize, and for MBEH to exert therapeutic effects on tumors away from the skin, a systemic application is needed.

MBEH has poor bioavailability and melanoma cells are less sensitive to the free pro-drug than melanocytes. This necessitates high amounts of MBEH to be administered. However, such high amounts are toxic for the subject. As a result, there is a need for means of delivery for the systemic administration of concentrated phenolic drugs, such as MBEH, to treat melanoma.

### BRIEF SUMMARY OF THE INVENTION

The field of the invention relates to nanoparticulate monobenzone that is useful for the treatment of melanoma. In particular, the field of the invention relates to nanoparticulate monobenzone comprising monobenzone or conjugates thereof that are cytotoxic and also generate an antitumor immune response. The field of the invention relates to the use of nanoparticulate monobenzone in pharmaceutical compositions for treating melanoma.

One aspect of the technology provides for nanoparticulate monobenzone. Nanoparticulate monobenzone comprises monobenzone or a conjugate thereof embedded within a nanostructured carrier. The nanostructured carrier can be a liposome or a metal-organic framework. Besides the monobenzone conjugate, the nanostructured carrier may further comprise a detectable label, a stabilizing agent, a targeting agent, linking agent, or any combination thereof and tailored for particular uses.

Monobenzone is a compound with the formula:

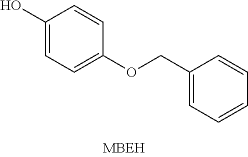 [SMILES: Oc1ccc(OCc2ccccc2)cc1]

Monobenzone conjugates may comprise an alkyl or aryl moiety covalently bound, directly or indirectly by a linking group, to monobenzone. Exemplary monobenzone conjugate include compounds of Formula I:

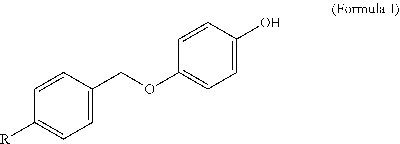

wherein R is an alkyl, aryl, —X-alkyl, or —X-aryl where X is a linking group such as —C(═O)—, —C(═O)O—, —C(═O)N(H)—, —O—, —N(H)—, or —N(alkyl)-.

Methods for the preparation of nanoparticulate monobenzone are also disclosed. The methods comprise embedding monobenzone or a conjugate thereof within the nanostructured carrier. In some embodiments, the nanostructured carrier is a liposome. In another aspect according to this disclosure, the nanostructured carrier is a metal-organic framework (MOF). When the nanostructured carrier is a liposome, the method may comprise the steps of preparing a composition comprising a lipid film and monobenzone or a conjugate thereof and forming a liposomal carrier comprising monobenzone or the conjugate thereof embedded within the liposomal carrier.

The disclosed monobenzone conjugates may be used to prepare and formulate pharmaceutical compositions for treating melanoma. As such, also disclosed herein are pharmaceutical compositions comprising an effective amount of any of the compounds or compositions disclosed herein, or pharmaceutically acceptable derivatives of any of the compounds or compositions disclosed herein, together with a pharmaceutically acceptable excipient, carrier, or diluent.

In some embodiments, the disclosed nanoparticulate monobenzone may be used for preparing a medicament for treating melanoma. In some embodiments, the disclosed nanoparticulate monobenzone may exhibit cytotoxicity and induce an immune response.

In some embodiments, the disclosed methods for treating melanoma may comprise administering to a subject in need thereof an effective amount of nanoparticulate monobenzone, or a pharmaceutical composition comprising an effective amount of the nanoparticulate monobenzone.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Topical MBEH prevents tumor growth. Tumor volumes in mice subcutaneously pretreated with 1.5 M topical MBEH or control (20% DMSO in 70% ethanol) for 3 weeks and challenged with 5×10⁵ B16-F10 mouse melanoma cells, *p<0.05.

FIG. 2. Melanocytes are undetectable in mouse skin topically treated with MBEH. TRP-1⁺ melanocytes (arrows) are detected in control animals but not in C57BL/6 skin treated with 1.5 M MBEH for 6 weeks.

FIG. 3. Melanosomal enzymes except MART-1 can mediate monobenzone cytotoxicity. Transfected HEK cells and B16-F10 melanoma cells were exposed to 250 μM of MBEH for 24 h before cytotoxicity was measured via MTT assay. All enzymes but MART-1 can mediate cytotoxicity to host cells. The data support the sensitivity of pigmented but also unpigmented melanoma cells by monobenzone.

FIG. 4. Lauroyl-MBEH-loaded nanoparticle uptake is favored by melanoma cells over normal tissue cells. B16 mouse melanoma cells and Ms22009 P2 normal mouse melanocytes were lifted from the flask and incubated with fluorescent (i.e., labeled with rhodamine PE), lauroyl-MBEH-loaded nanoparticles for 60 minutes. Efficient uptake by tumor cells is responsible for the uptick in fluorescence within B16 cells but not normal melanocytes, demonstrating the selectivity of nanoparticulate MBEH application for malignant cells.

FIG. 5. Tumors preferentially take up circulating nanoparticles in vivo. FACS analysis of 1 μm FITC-labeled liposomes administered intravenously 24 h prior, into mice subcutaneously challenged with 14-day B16-F10 tumors. At n=2, fluorescence of tissue homogenates was compared to tissues from control mice not exposed to liposomes.

FIG. 6. CD8 T cell infiltration in tumors is enhanced following MBEH exposure. The number of cytotoxic T cells was 4.7-fold enhanced after MBEH exposure (n=3, Student's t-test, p<0.01) in B16-F10 tumors in mice pretreated topically (a) without MBEH or (b) with 1.5 M of MBEH. Example cross sections of remaining tumor tissue immunostained for CD8 are shown.

FIG. 7. The design of exemplary lauroyl-MBEH-loaded liposomes for selective delivery to melanoma tumors. In the first step, MBEH conjugate and lipids, such as dipalmitoylphosphatidylcholine (DOPC), are extruded together to form the parent lauroyl-MBEH-loaded liposomes. Subsequent conjugation of a stabilizing agent, such as polyethylene glycol (PEG), may optionally be performed to increase the stability of nanoparticulate monobenzone. Alternatively or additionally, the nanoparticular monobezone may be modified with a linking agent, such as cholesterol-PEG(2K)-DBCO (DBCO=dibenzocyclooctyl), which allows for both increased stability and ability to conjugate with one or more additional liposomal components such as a targeting agent or detectable label. Exemplary targeting agents include antibodies to melanoma surface antigens, such as ganglioside precursor disialohematoside (GD3) for further enhanced selectivity. Successful loading of MBEH into liposomal nanoformulations is demonstrated by ICP and UV-vis data of digested/dissolved nanoparticles.

FIG. 8. Viability impact at ˜10-fold lower molar concentrations for the lauroyl-MBEH-loaded liposomes than free MBEH in 24 h. Free MBEH or lauroyl-MBEH-loaded liposomes were added to B16 melanoma cells in culture. After 24 h, the viability among the remaining cells was measured at different nanoparticle concentrations. With half-maximum cytotoxicity (IC₅₀) concentration at 50 μM MBEH for the lauroyl-MBEH-loaded liposomes, there is a minimal 10-fold advantage to the free drug (IC₅₀˜ 500 μM). The two panels show data from two different experiments using two different batches of the lauroyl-MBEH-loaded liposomes.

FIG. 9. First panel: Single cell functional proteomics data indicates functional heterogeneity when comparing the effects of free and nanoparticulate MBEH on cellular functions among live cells. Adherent M14 melanoma cells were incubated for 24 h with 100 μM of free MBEH (a), nanoparticulate monobenzone with lauroyl MBEH-loaded liposomes (b), MBEH-loaded MOF nanoparticles (c) or vehicle alone (d) before being harvested and loaded into individual wells of the respective Isocode chips (˜30,000 cells/sample) to measure the presence of phosphorylated proteins among individual, live cells by IsoPlexis functional proteomics. Single-cell activation profiles for live M14 melanoma cells exposed to MBEH-loaded MOF NPs for 24 h, show notable decreases in protein phosphorylation for DNA-repair, cytokine-release, and cell-proliferation pathways in comparison to cells that have not been exposed (i.e., vehicle only). The decreases in cell-proliferation pathways are even more than those observed in cells that have been exposed to the free MBEH. The individual phosphoproteins measured (results are shown for ˜300 live, single cells per group) are categorized as shown in the legend. The most profound effect on cell signaling was found in presence of long-tailed MBEH. Over the treatment period, untreated cells gained 96% viable cells, whereas cell numbers were reduced by 45, 29 and 1% respectively in response to the free drug, MBEH-loaded MOFs and liposomes loaded with lauroyl MBEH, respectively. Functional heterogeneity among treatments was driven by PARP, P-eIF4α and ribosomal P-S6. Second panel: Viability changes of M14 melanoma cells shown between 24-48 h of treatment. M14 cells were treated with 100 μM free MBEH; 100 μM liposomal lauroyl MBEH*; 100 μM MBEH-loaded UiO66**; and control (i.e., vehicle). (*Not PEGylated, ** Not liposomal)

FIG. 10. Dynamic light-scattering (DLS) traces of the as-synthesized DOPC liposomes.

FIG. 11. DLS traces of the lauroyl-MBEH-loaded DOPC liposomes.

FIG. 12. DLS traces of the Rhodamine-PE-labeled, lauroyl-MBEH-loaded liposomes.

FIG. 13. (a) The structure of UiO66 metal-organic framework includes the Zr clusters showing that the phenolic oxygen of MBEH can coordinate to a Zr site. Additionally, MBEH can be located within the pores of the framework to form MBEH-loaded UiO66. (b) Microscopy shows the size and general shape of a sample of UiO66 MOF particles.

FIG. 14. Compared to a control, cell viability is reduced when exposed to MBEH-loaded UiO66. Representative images and a plot showing reduced viability when B16-F10 cells are exposed to UiO-66 nanoparticles loaded with 100 mM MBEH for 24 h.

### DETAILED DESCRIPTION OF THE INVENTION

Disclosed herein are nanoparticular monobenzone compositions for use in the treatment of melanoma. Malignant melanoma is the most aggressive form of skin cancer and is responsible for most skin-cancer deaths. Although immunotherapeutics have recently been put to use as highly promising treatments, complete responders are still rare due to the high propensity for melanoma to metastasize and escape. Thus, there is a critical need for new drug candidates that can bring metastatic melanoma into total remission through novel strategies. Substituted phenols can target the melanogenic pathway, the primary mechanism for producing melanin pigment inside pigment-producing cells. In this pathway, the amino acid L-tyrosine is first oxidized to dopa-quinone by the enzymes tyrosinase and tyrosinase-related protein-1 (TRP-1), abundantly present in melanosomes, lysosome-like organelles that are uniquely present in melanocytes and melanoma cells. Aborting full maturation to melanin using phenol prodrugs that are structurally similar to tyrosine or intermediates of the melanogenic pathway as alternate substrates for melanogenic enzymes can result in reactive quinone products that are toxic to the host cells

Several factors have thus far prevented the translation of MBEH into systemic treatments. First, MBEH is highly insoluble in water, resulting in very low bioavailability. Second, melanoma cells are less sensitive to treatment with the free drug than melanocytes, attributed in part to the ability of tumor cells to purge toxic compounds through efflux transporters, upregulated during malignant transformation. Third, high concentrations of free MBEH that are toxic to the host would be necessary to mediate melanoma regression when administered systemically.

The nanoparticulate monobenzone composition described herein can overcome the aforementioned challenges by reducing the amount of the active ingredient necessary to inhibit the growth and proliferation of melanoma cells. The nanoparticulate monobenzone compositions described herein comprise nanostructured carriers embedded with substituted phenol active ingredients, such as MBEH or conjugates thereof. They are also more favorably taken up by tumors than by normal tissue cells; and their drug contents are preferentially released in the context of acidic melanosomes, thereby maximizing the prodrug exposure to melanogenic enzymes. This can result in a significant reduction in the amount of active ingredient necessary to demonstrate inhibition of growth or proliferation of tumor cells.

The term “nanostructured carrier” as contemplated herein means a carrier having dimensions less than a micron. Nanostructured carriers may have dimensions between 30 to 800 nm, 30 to 500 nm, 30 to 200 nm, 30 to 120 nm, 30 to 100 nm, or 30 to 80 nm. In some embodiments, the nanostructured has dimensions of 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 nm, or any range therebetween.

The nanostructured carriers can be loaded with MBEH or conjugates thereof. The hydrophobic drugs may comprise between 1-50 mol %, such as between 5-25 mol % or 10 to 25 mol %, of the lipid content in the nanostructured carrier. In some embodiments, MBEH or a conjugate thereof comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 mol %, or any range therebetween of the lipid content in the nanostructured carrier. In some examples, the nanoparticulate monobenzone were synthesized with 1, 2.5, 5, 10, or 15 mol % of MBEH.

MBEH (monobenzone) is a compound with the formula

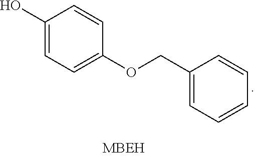 [SMILES: Oc1ccc(OCc2ccccc2)cc1]

Other substituted phenols may be utilized in the compositions and methods described herein provided that they can be converted by tyrosinase to a product that is toxic to the host cells. In some embodiments, monobenzone conjugates are utilized in the compositions and methods described herein. As used herein, monobenzone conjugates comprise may comprise an alkyl or aryl moiety covalently bound, directly or indirectly by a linking group, to monobenzone, such as at the benzyl ether portion of monobenzone. The alkyl or aryl moiety may be in para-position relative to the methyl ethyl linking the phenyl to the phenol but may also be in ortho- or meta position as well. Exemplary monobenzone conjugates include compounds of Formula I:

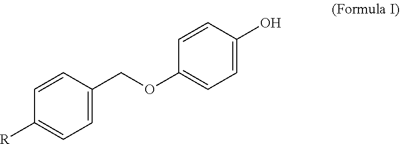

wherein R is an alkyl, aryl, —X-alkyl, or —X-aryl where X is a linking group such as —C(═O)—, —C(═O)O—, —C(═O)N(H)—, —O—, —N(H)—, —N(alkyl)-.

The term “alkyl” as contemplated herein means a saturated or unsaturated, e.g., alkenyl or alkynyl, hydrocarbon radical in all of its isomeric forms, such as a straight or branched group. The term “alkenyl” as used herein refers to an unsaturated straight or branched hydrocarbon having at least one carbon-carbon double bond. The term “alkynyl” as used herein refers to an unsaturated straight or branched hydrocarbon having at least one carbon-carbon triple bond. In some embodiments, the alkyl may have between 1-28, 4-28, 6-22, 8-18 carbon atoms, which may be referred to as a C₁-C₂₈, C₄-C₂₈, C₆-C₂₂, or C₈-C₁₈ alkyl, respectively. In particulate embodiments, the alkyl group comprises 1, 2, 3, 4, 5, 6, 7, 8, 9, 10, 11, 12, 13, 14, 15, 16, 17, 18, 19, 20, 21, 22, 23, 24, 25, 26, 27, or 28 carbon atoms or any range therebetween.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido or carboxamido, carboxylic acid, —C(O)alkyl, —CO₂alkyl, carbonyl, carboxyl, carboxyamido, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The alkyl or aryl groups may be selected to facilitate the loading of the monobenzone conjugate. This may include increased the amount of monobenzone conjugate that can be embedded within the nanostructured carrier. In some embodiments, the alkyl may be structurally similar to a fatty acid in number of carbon atoms, number of saturated or unsaturated carbon-carbon bonds, positioning of unsaturated carbon-carbon bonds. Exemplary fatty acids which the alkyls may bear structural similarity to include, without limitation, caprylic, capric, lauric, myristic, palmitic, stearic, arachidic, behenic, lignoceric, cerotic, myristoleic, palmitoleic, sapienic, oleic, elaidic, vaccenic, linoleic, linolaidic, α-linolenic, arachidonic, eicosapentaenoic, erucic, or docosahexaenoic acid.

Monobenzone conjugates may be prepared by the addition of hydroquinone to a substituted (bromomethyl)phenyl. For example, lauroyl MBEH (compound 3) may be prepared by adding potassium carbonate and 1-(4-(bromomethyl)phenyl)-1-dodecanone (compound 2) to a solution of hydroquinone. Exemplary methods for preparing monobenzone conjugates are provided in the Examples. Those of ordinary skill will appreciate that other synthetic methods may be used to prepared monobenzone conjugates.

Exemplary conjugated monobenzone include lauroyl MBEH and lauryl MBEH, which have the structures shown below.

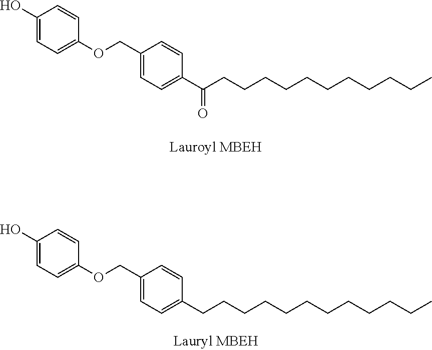 [SMILES: CCCCCCCCCCCC(=O)c1ccc(COc2ccc(O)cc2)cc1 ; CCCCCCCCCCCCc1ccc(COc2ccc(O)cc2)cc1]

In some embodiments, the nanostructured carrier is a liposomal carrier or liposome. Liposomes can be classified as unilamellar vesicles (ULVs), oligolamellar vesicles (OLVs), multilamellarvesicles (MLVs), and multivesicular liposomes (MVLs), depending on the compartment structure and lamellarity. ULVs can be further divided into small unilamellar vesicles (SUVs, 30-100 nm), large unilamellar vesicles (LUVs, >100 nm), and giant unilamellar vesicles (GUVs, >1000 nm). Liposomal carriers may be prepared that are highly biocompatible, easily modified, and tailored for specific applications. In one non-limiting example for use with MBEH conjugates, the liposomal carrier can be ˜100 nm diameter.

Liposomal carriers comprise a lipid bilayer that may be prepared from one or more amphiphilic components. The amphiphilic components may include a glycerolphospholipid (GP) and/or sphingomyelin (SM). The hydrophilic head group of the amphiphilic components may comprise phosphatidylcholine (PC), phosphatidyl ethanolamine (PE), phosphatidyl serine (PS), phosphatidyl inositols (PI), phosphatidic acid (PA), phosphatidylglycerol (PG), cardiolipin, or the like. The hydrophobic groups of the amphiphilic components may be selected from fatty acids, such as decanoic acid, lauric acid, palmitic acid, oleic acid, myristic acid, stearic acid, erucic acid, and the like, or sphingosine.

GPs and SM play a key role in formulation since they affect the biophysical properties of liposomes (e.g., drug encapsulation, stability, and drug release) and further influence the pharmacokinetic behavior and pharmacodynamics in vivo. The length, symmetry, inter- and intra-molecular interactions, branching, and unsaturation degree of hydrocarbon chains decide the thickness and fluidity of the bilayer, phase transition temperature, and drug release rate. Longer hydrocarbon chains can induce a tighter membrane packing and increase drug retention. A higher degree of unsaturation or branching of the hydrocarbon chain could result in looser membrane packaging.

Exemplary amphiphilic components include, without limitation, HSPC (hydrogenated soy phosphatidylcholine); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phosphoethanolamine); DPPC (1,2-dipalmitoyl-sn-glycero-3-phosphocholine), DODAP (1,2-dioleoyl-3-dimethylammonium propane), DOTAP (1,2-dioleoyl-3-trimethylammonium propane), and DPGG (1,2-dipalmitoyl-sn-glycero-3-galloyl), etc.

Steroids, such as cholesterol or cholesterol derivatives, may also be included in the liposomal carrier. Steroids, such as cholesterol, can promote the packing of lipid chains and bilayer formation, modulate the fluidity/rigidity of membrane, affect the drug release, stability of liposomes, and the kinetics of exocytosis. Optionally, the steroid may be conjugated, directly or indirectly, to another component of the liposomal carrier. The conjugated component may include a stabilizing agent, detectable label, targeting agent, or linking group.

Liposomal carriers can be prepared with detectable labels for detection of liposomal trafficking. Exemplary detectable labels, include, without limitation, fluorescent labels, near-IR labels, X-ray-active labels, radioactive labels, and magnetic-resonance-imaging labels.

Liposomal carriers can be prepared with stabilizing agents. To improve liposome stability and enhance their circulation times in the blood, sterically stabilized liposomes may be prepared. A hydrophilic polymer, such as polyethylene glycol (PEG), can be used to prepare sterically stabilized liposomes. The establishment of a steric barrier improves the efficacy of encapsulated agents by reducing in vivo opsonization with serum components, and the rapid recognition and uptake by the reticuloendothelial system. This reduces the elimination of drugs by prolonging blood circulation and providing accumulation at pathological sites while also attenuating side effects such as polyethylene glycol (PEG) oligomers on the exterior of the bilayer. Such PEG groups can include MPEG (methoxy polyethylene glycol). Alternatively, stabilizing agents such as steroids can be used to stabilize the lipid bilayer by embedding within the bilayer.

The outer surface of liposomal carriers can include targeting agents. Ligand-targeted liposomes offer potential for site-specific delivery of drugs to designated cell types or organs in vivo, which selectively express or over-express specific ligands (e.g., receptors or cell-adhesion molecules) at the site of disease. Many types of ligands are available, such as antibodies, peptides/proteins, carbohydrates, oligonucleotides such as DNA, RNA, small molecules, or combinations thereof. The coupling of antibodies, particularly monoclonal antibodies, to create immunoliposomes represents a versatile ligands that can be affixed to liposome surfaces

The liposomal nanocarrier may comprise a linking agent. As used herein, a “linking agent” is a compound or liposomal component having a functional group that is readily functionalized via a conjugation strategy generally suitable for biological molecule. Exemplary functional group include those that can participate via an amine reaction (e.g., NHS ester, imidoester, hydroxymethyl phosphine, guanidination of amine, fluorophenyl esters, carbodiimides, anhydrides, arylating agents, carbonates, aldehydes or glyoxals), thiol reactions (e.g., maleimide, haloacetyl, pyridyldisulfide, vinyl sulfone, or thiol-disulfide exchange), carboxylate reactions (e.g., carbodiimides), hydroxyl reactions (e.g., isocyanates, enzymatic oxidation, or carbonyldiimidazole), aldehyde and ketone reaction (e.g., hydrazine derivative, Schiff-base formation, or reductive amination), active hydrogen reaction (e.g., iodination reaction), photo-chemical reactions (e.g., psoralen compounds, aryl azides and halogenated aryl azides, benzophenones, anthraquinones), or cycloaddition reactions (e.g., chemoselective ligation such as click chemistry or Diels-Alder reaction).

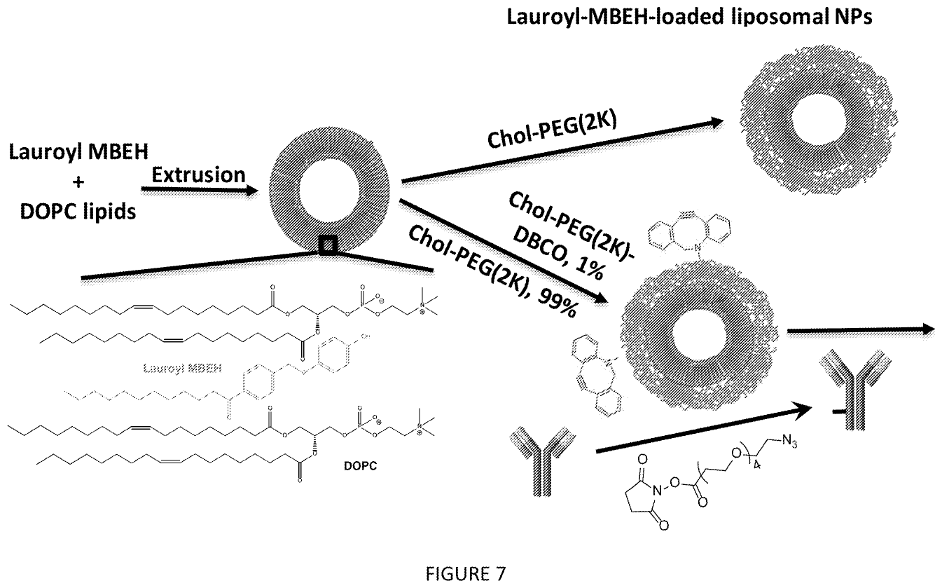

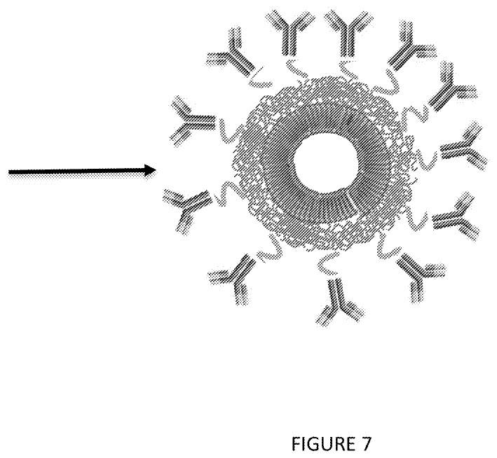

FIG. 7 illustrates exemplary modifications to the liposomal nanocarriers described herein. As demonstrated by example, the surface of a liposomal nanocarrier, which may contain lauroyl MBEH, can be modified with a stabilizing agent, such as cholesterol-PEG(2000) (Chol-PEG(2K)), to prolong their stability in circulation and prevent opsonization. The Chol-PEG(2K) can be incorporated onto the surface of the liposomes post-extrusion (FIG. 7, 2^{nd} step in top pathway). To enhance the specificity for melanoma cells, lauroyl-MBEH-loaded NPs can be modified with a targeting agent, such as antibody R24 to the melanoma surface antigen GD3. In general, the NPs can be functionalized via any suitable conjugation strategy. An exemplary conjugation strategy involves click chemistry reactions. As exemplified by FIG. 7, the liposomal nanocarrier may be functionalized with commercially available Chol-PEG(2K)-DBCO (FIG. 7, 2^{nd} step, bottom pathway), and then conjugated to the azide-modified antibodies¹⁵ using Cu-free click chemistry (FIG. 7, 3^{rd} step, bottom pathway).

In other embodiments, the nanostructure carrier is a metal-organic framework (MOF) nanoparticle. MOFs are a class of compounds comprising of metal ions or clusters (i.e., nodes) coordinated to multitopic organic ligands (i.e., linkers) to form multidimensional structures. They are a subclass of coordination polymers, with the special feature that they are often porous when assembled into a three dimensional structure. MOFs may be prepared to have low toxicity and/or be biocompatible. MOF-based nanostructured may be loaded with MBEH or a conjugate thereof and the MBEH or MBEH conjugate may be released within the subject.

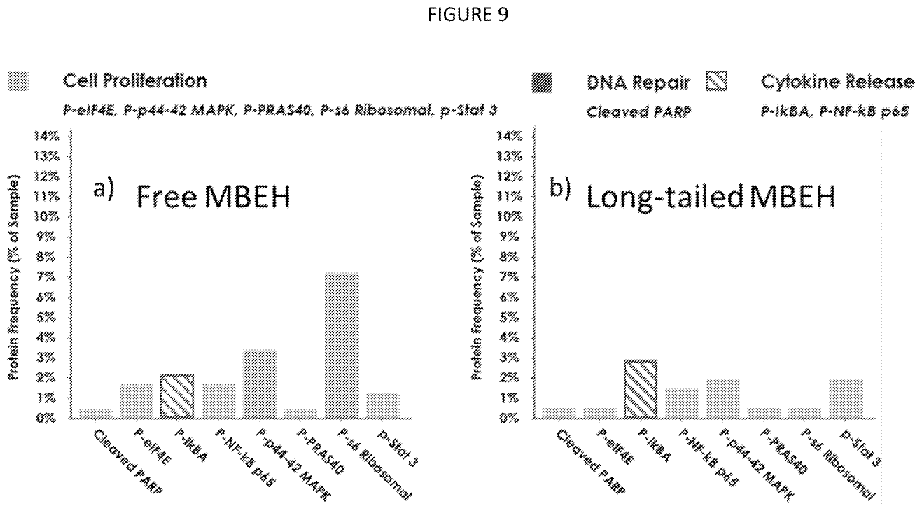

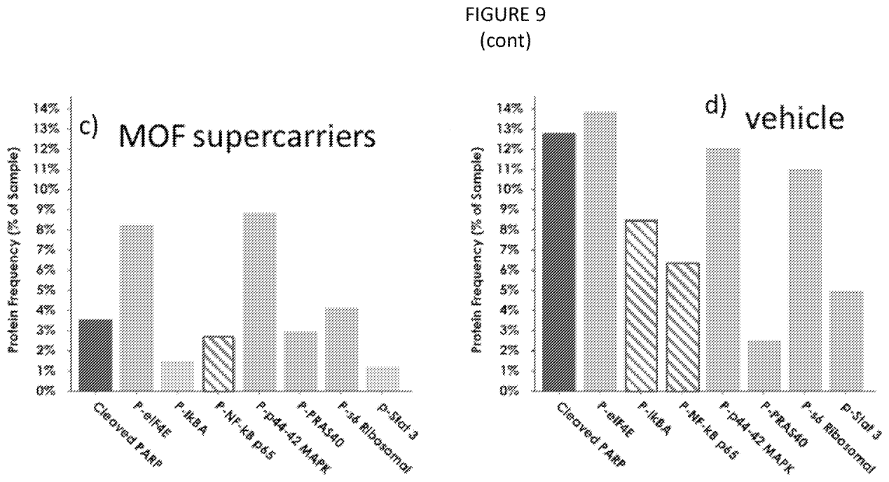

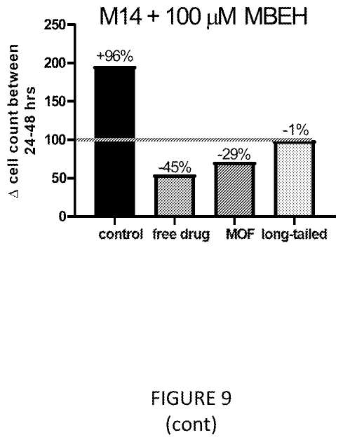

An exemplary MOF for use as a nanostructured carrier is the zirconium-based metal-organic framework UiO-66 (FIG. 9), which was utilized as a nanostructured carrier for the systemic delivery of MBEH or MBEH conjugates. To optimize the loading of UiO66 with high mass of MBEH, nanoparticle formation can involve self-assembly or encapsulation. A preferred strategy is to expose the MOF NPs to a solution of the drug, allowing for the drug molecules to infiltrate into the pores of the MOF NPs and/or to bind to the nodes/linkers of the MOF, effectively loading the MOF NPs with the drug molecules.

Methods of treating subjects in need of nanoparticulate monobenzone are also provided. Nanoparticulate monobenzone may be administered to the subject by any suitable route. In some embodiments, the nanoparticulate monobenzone is systemically administered Nanoparticulate monobenzone may be administered parenterally, e.g., injection, infusion or implantation, or enterally, e.g., orally.

As used herein, a “patient” may be interchangeable with “subject” or “individual” and means an animal, which may be a human or non-human animal, in need of treatment. Suitable patients for the disclosed methods may include, for example mammals, such as humans, monkeys, dogs, cats, horses, rats, and mice. Suitable human patients include, for example, those who have melanoma or those who have been determined to be at risk for developing melanoma, or have related disorders. As used herein, a “subject in need of treatment” may include a patient having melanoma that is responsive to therapy with nanoparticulate monobenzone described herein.

As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of melanoma. As such, the methods disclosed herein encompass therapeutic administration. In some embodiments, treatment may result in the killing of cancer cells, such as melanoma tumor cells, or inhibition of the growth or proliferation of cancer cells. In some embodiments, treatment may result in increased T cell infiltration.

As used herein the term “effective amount” refers to the amount or dose of the nanoparticulate monobenzone, upon single or multiple dose administration to the subject, which provides the desired effect in the subject under diagnosis or treatment. The disclosed methods may include administering an effective amount of the disclosed nanoparticulate monobenzone (e.g., as present in a pharmaceutical composition) for treating melanoma, whereby the effective amount causes the killing of cancer cells, inhibition of the growth or proliferation of cancer cells, or increases T cell infiltration in the patient.

An effective amount can be readily determined by the attending diagnostician, as one skilled in the art, by the use of known techniques, and by observing results obtained under analogous circumstances. In determining the effective amount or dose of nanoparticulate monobenzone administered, a number of factors can be considered by the attending diagnostician, such as: the species of the subject; its size, age, and general health; the degree of involvement or the severity of the disease or disorder involved; the response of the individual patient; the particular compound administered; the mode of administration; the bioavailability characteristics of the preparation administered; the dose regimen selected; the use of concomitant medication; and other relevant circumstances.

The pharmaceutical compositions for use according to the methods as disclosed herein may include a single nanoparticulate monobenzone as an active ingredient or a combination of nanoparticulate monobenzones as active ingredients (e.g., a combination of nanoparticulate monobenzone having different labels, loadings of monobenzone conjugates, stabilizing or targeting agents). For example, the methods disclosed herein may be practiced using a composition containing nanoparticulate monobenzone with a single type of monobenzone conjugate. Alternatively, the disclosed methods may be practiced using a composition containing a nanoparticulate monobenzone with two or more types of monobenzone conjugates. In another non-limiting example, two or more nanoparticulate compositions differing in their loading of monobenzone conjugate, labels, stabilizing agents, or targeting agents can be combined.

The disclosed methods may be practiced by administering a first pharmaceutical composition comprising nanoparticulate monobenzone and administering a second, different pharmaceutical composition (e.g., a pharmaceutical composition comprising a different nanoparticulate monobenzone or different active pharmaceutical ingredient), where the first composition may be administered before, concurrently with, or after the second composition. As such, the first pharmaceutical composition and the second pharmaceutical composition may be administered concurrently or in any order, irrespective of their names.

As one skilled in the art will also appreciate, the disclosed pharmaceutical compositions can be prepared with materials (e.g., actives excipients, carriers, and diluents etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

Pharmaceutical compositions comprising the nanoparticulate monobenzone may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal injection) route. Such formulations may be prepared by any method known in the art of pharmacy, for example by bringing into association the active ingredient with the carrier(s) or excipient(s).

The nanoparticulate monobenzone utilized in the methods disclosed herein may be formulated as a pharmaceutical composition in liquid dosage form, although any pharmaceutically acceptable dosage form can be utilized. Exemplary liquid dosage forms include, but are not limited to pharmaceutical compositions for parenteral administration, such as injectable liquids, injectable emulsions, or gels.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets

Alternatively, the nanoparticulate monobenzone utilized in the methods disclosed herein may be formulated as a pharmaceutical composition in solid dosage form. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended-release dosage form, pulsatile release dosage form, mixed immediate release and controlled release dosage form, or a combination thereof.

The nanoparticulate monobenzone utilized in the methods disclosed herein may be formulated as a pharmaceutical composition that includes an excipient, carrier, or diluent. For example, the excipient, carrier, or diluent may be selected from a group consisting of buffered physiologic salt solutions suitable for injection.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term that are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Activity of Nanoparticulate Monobenzone

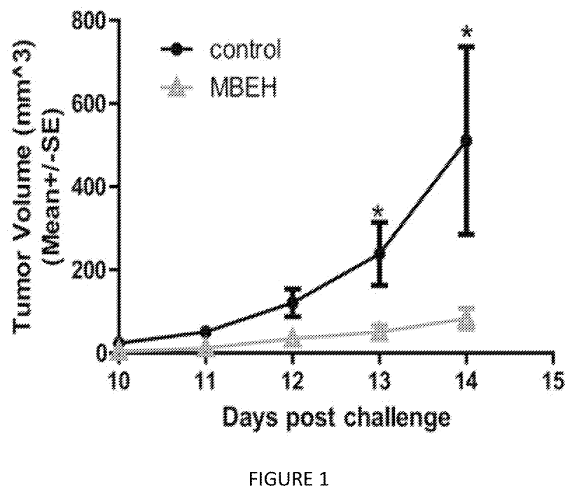

Monobenzyl ether of hydroquinone (MBEH), a phenolic compound and an FDA-approved drug, has been found to reduce subcutaneous melanoma growth (FIG. 1), not only by inducing necrosis in pigmented cells but also by promoting a dense accumulation of CD8 T cells in the tumors, essentially generating antitumor immune responses that operate synergistically along with its native cytotoxicity that allows for treatment for melanoma that combines both chemo- and immunotherapeutic effects.

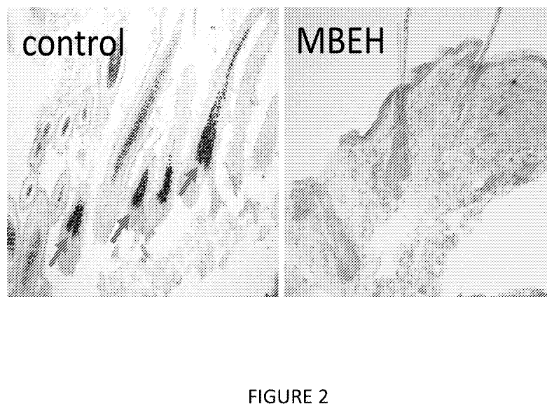

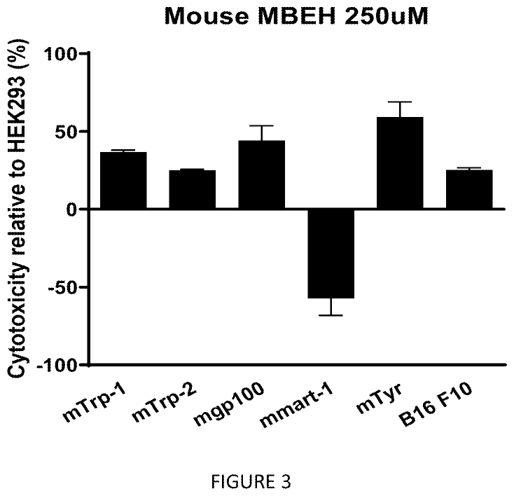

As an FDA-approved treatment to induce skin depigmentation (FIG. 2) in patients with vitiligo, MBEH is safe as a topically applied formulation. When applied to keratinocytes and melanocytes in culture, it shows remarkable selectivity in targeting the latter. This explains its bleaching effect, and subsequent melanocyte death, upon topical application without inducing further skin damage. In addition to this innate selectivity for pigmented cells, an MBEH-based systemic treatment for melanomas should also be attractive for amelanotic tumors because other melanosomal proteins TRP-1, TRP-2, and gp100 (but not MART-1) can also mediate MBEH toxicity in addition to tyrosinase (FIG. 3).

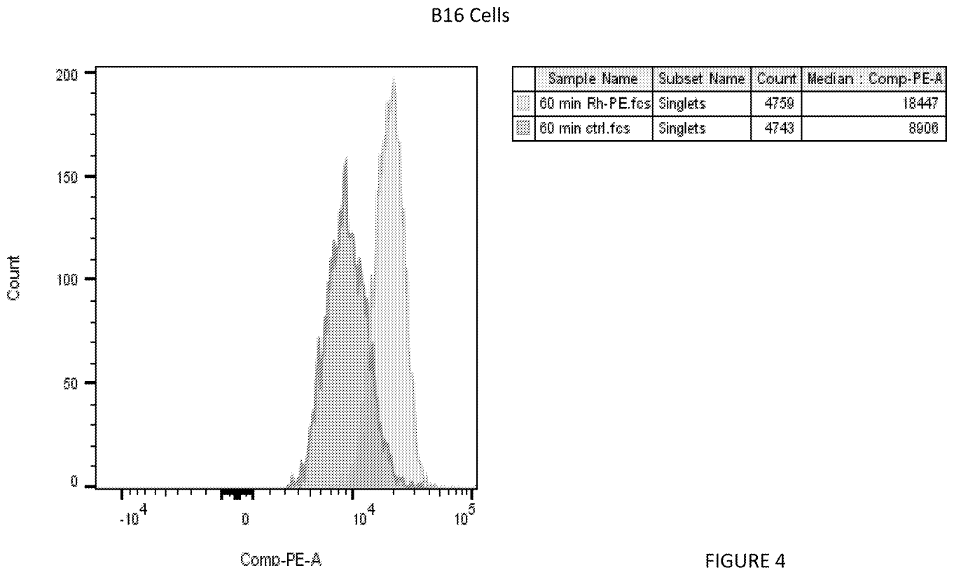

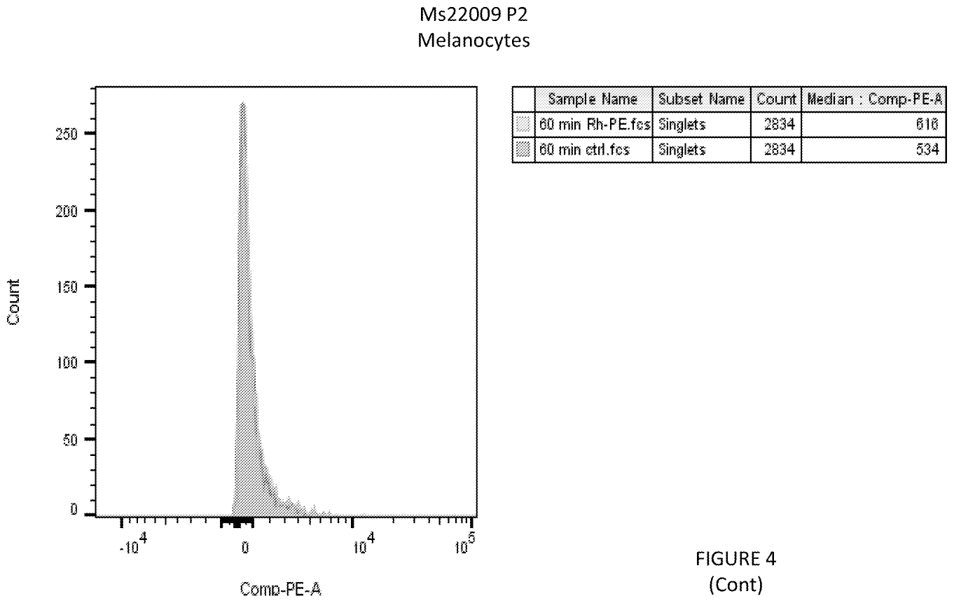

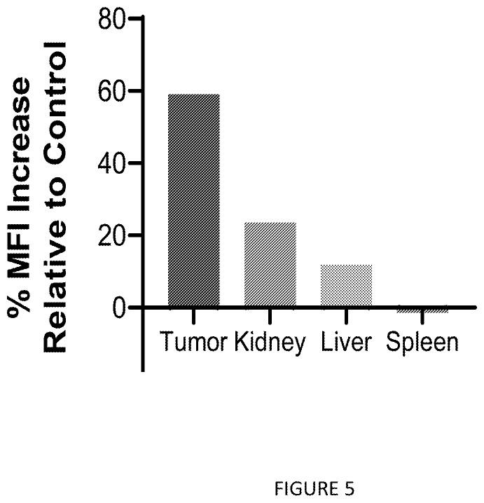

As demonstrated in the Examples, 100 nm liposomes are favorably taken up by tumor cells over normal tissue cells (FIG. 4). Liposomal carriers are also found to accumulate primarily in melanoma-derived tumors in mice after intravenous administration (FIG. 5), confirming that these nanostructured carriers can be excellent candidate carriers for MBEH conjugates. Thus, similar liposomal NPs, when loaded with MBEH or an MBEH conjugate, can be preferentially taken up by melanoma tumors through interactions with the lipid bilayers and subsequent endocytosis. The resulting endosomes can fuse with lysosomes and melanosomes,^{10, 11} to release a large dose of MBEH or MBEH conjugate within organelles laden with melanosomal enzymes.

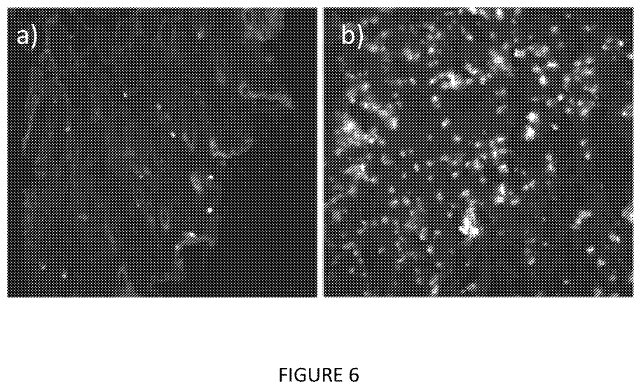

Chemotherapeutically, MBEH, MBEH conjugates, and other substitute phenols can target the melanogenic pathway, resulting in necrotic cell death of melanogenic cells. Experiments with C57BL/6 mice have also shown that topical application of MBEH prior to subcutaneous tumor challenge results in remarkable T cell infiltration into the tumors after two weeks (FIG. 6). Together, these observations suggest that the development of liposomal NPs loaded with MBEH or MBEH conjugates such as lauroyl MBEH, aka “long-tailed” MBEH (FIG. 7) can be a two-pronged systemic treatment for melanoma, leading to new treatments that can bring about total remission for melanoma patients. This approach takes advantage of the specific cytotoxicity of the parent MBEH drug through the melanogenic pathway, which is highly selective for melanoma, preferential uptake of lauroyl-MBEH-loaded liposomal NPs by melanoma tumor cells, and the selective trafficking to relevant melanosomal compartments. This approach also capitalizes on synergistic immune activation against tumor cells that did not succumb to the drug. This leads to enhanced outcomes over just chemotherapeutical alone.

Lauroyl-MBEH-loaded liposomes were synthesized with ˜10 mol % lauroyl MBEH incorporated into the DOPC lipid bilayer following previously established protocols¹² and are stable at 4° C. in 20 mM HBS buffer (FIG. 7, 1^{st} step) for up to 3 months. The synthesis can be carried out with 1 mol % of 18:1 Liss Rhodamine PE (Rhod-PE) lipid as a fluorescent tag to correlate the observed cytotoxicity with: 1) cellular uptake of the liposomal NPs and 2) their intracellular trafficking in cells in vitro or tumor tissue in vivo using fluorescent-activated cell scanning (FACS).

The surface of the lauroyl-MBEH-loaded liposomal NPs can be modified with cholesterol-PEG(2000) (Chol-PEG(2K)) to prolong their stability in circulation and prevent opsonization. The Chol-PEG(2K) can simply be incorporated onto the surface of the liposomes post-extrusion (FIG. 7, 2^{nd} step, top pathway). To enhance the specificity for melanoma cells, lauroyl-MBEH-loaded NPs can be modified with antibody R24 to the melanoma surface antigen GD3. The NPs will first be functionalized with commercially available Chol-PEG(2K)-DBCO (FIG. 7, 2^{nd} step, bottom pathway), and then conjugated to the azide-modified antibodies¹⁵ using Cu-free click chemistry (FIG. 7, 3^{rd} step, bottom pathway).

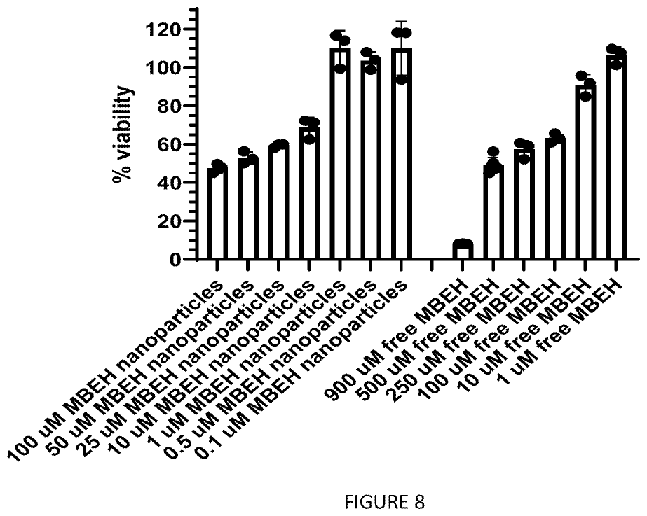

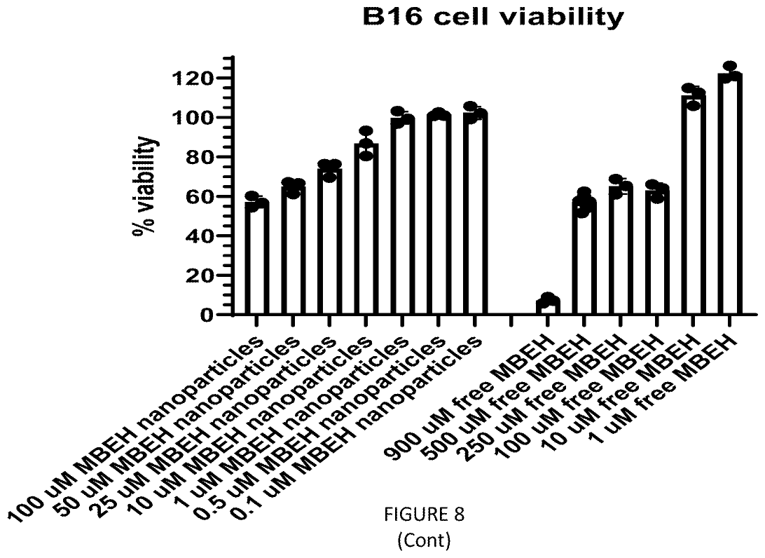

The parent lauroyl-MBEH-loaded liposomal material ((FIG. 7, 1^{st} step) exhibited much greater cytotoxicity than the unincorporated MBEH drug (FIG. 8). In addition, IsoPlexis single-cell functional proteomics data for M14 melanoma cells exposed to the lauroyl-MBEH-loaded NPs showed reduced tendency for individual cells to repair DNA, release cytokines, and proliferate, as indicated by the reduced presence of the corresponding phosphorylated-signaling proteins in comparison to the non-treated cells (FIG. 9). Last. exposing B16-F10 melanoma cells and mouse melanocytes to the Rhod-PE-labeled, lauroyl-MBEH-loaded liposomes clearly showed faster uptake of the NPs by the cancer cells (FIG. 4). Together, these data indicated that lauroyl-MBEH-loaded liposomes, or other MBEH or MBEH conjugate loaded nanocarriers, can be used in a method for treating melanoma.

### Synthesis of Lauroyl MBEH

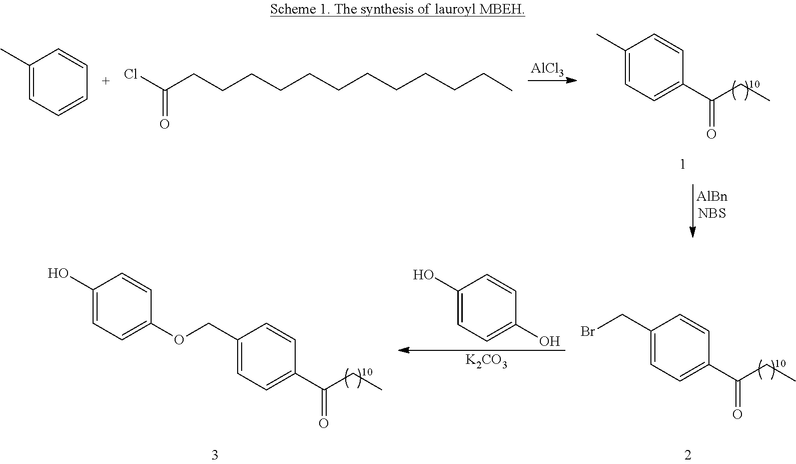

Synthesis and Characterization of 1-(p-Tolyl)-1-Dodecanone (Compound 1).

This synthesis was adapted from a published procedure for the synthesis of the tridecanoyl analog. [Yu P, Hu J, Zhou T-Y, Wang P, Xu Y-H. Synthesis, insecticidal evaluation of novel 1,3,4-thiadiazole chrysanthemamide derivatives formed by an EDCl/HOBt condensation. J. Chem. Res. 2011; 35(12):703-706] To a mixture of toluene (21.5 g, 234 mmol, 18 equiv) and aluminum trichloride (7.33 g, 55 mmol, 4.2 equiv) was added lauroyl chloride (2.84 g, 13 mmol, 1 equiv) dropwise in the ice bath. The reaction mixture was heated to reflux and stirred for overnight. After completion of the reaction, the mixture was poured into ice water. The pH of the mixture was adjusted to 5-6 with concentrated hydrochloric acid and extracted with benzene (3×30 mL). Inside a separatory funnel, the combined organics was successively washed with 10% aqueous sodium bicarbonate until the pH of the aqueous layer reached ˜7 and then with DI water. The isolated organic layer was dried over anhydrous MgSO₄, filtered, and concentrated on a rotary evaporator to afford compound 1 as white crystals (2.7 g, 76% yield).

The structure and composition of compound 1 was confirmed by NMR spectroscopy and mass spectrometry. ¹H NMR (500 MHz, CDCl₃): δ 7.86 (2H, d, J=8.2 Hz), 7.25 (2H, d, J=8.0 Hz), 2.92 (2H, t, J=7.4 Hz), 2.40 (3H, s), 1.75-1.69 (2H, m), 1.26 (16H, br), 0.88 (3H, t, J=6.9 Hz). ¹³C NMR (126 MHz, CDCl₃): δ 200.3, 143.6, 134.7, 129.2, 128.2, 38.5, 31.9, 29.6, 29.6, 29.5, 29.5, 29.43, 29.36, 24.5, 22.7, 21.6, 14.1. MS (ESI): Exact Mass Calcd for [C₁₉H₃₁O]+ (M+H+)=275.2. Found 275.2.

### Synthesis and Characterization of 1-(4-(Bromomethyl)Phenyl)-1-Dodecanone (Compound 2).

The synthesis of compound 2 was adapted from a published procedure for the synthesis of 1-(4-(bromomethyl)phenyl)propan-1-one. [Yang Y, Yu Y, Li X, Li J, Wu Y, Yu J, Ge J, Huang Z, Jiang L, Rao Y. Target elucidation by cocrystal structures of NADH-ubiquinone oxidoreductase of Plasmodium falciparum (PJNDH2) with small molecule to eliminate drug-resistant malaria. J. Med. Chem. 2017; 60(5):1994-2005] To a solution of compound 1 (2.72 g, 9.9 mmol, 1.0 equiv) in anhydrous benzene (20 mL) in a 100 mL round-bottom flask equipped with a magnetic stir bar were added 1.98 g (11.1 mmol, 1.1 equiv) of N-bromosuccinimide (NBS) and 168.8 mg (1.0 mmol, 0.1 equiv) of 2,2′-azobis(2-methylpropionitrile) (AIBN). A water-cooled reflux condenser was attached to the flask and the reaction mixture was placed under argon and heated to 70° C. when an aliquot of anhydrous MeCN (7 mL) was added to completely dissolve NBS. The temperature of the reaction mixture was then raised to 90° C. and kept refluxed at this temperature for 7 h. After cooled down, the reaction mixture was evaporated to dryness, redissolved in DCM (90 mL), and washed with DI water (90 mL). The organic layer was dried over anhydrous MgSO₄, filtered, and evaporated to dryness before being purified by silica gel flash column chromatography (hexanes/EtOAc=30:1 v/v). The crude product was recrystallized in hexanes to yield compound 2 as white crystals (1.36 g, 39% yield).

The structure and composition of compound 2 was confirmed by NMR spectroscopy and mass spectrometry. ¹H NMR (500 MHz, CDCl₃): δ 7.94 (2H, d, J=8.2 Hz), 7.48 (2H, d, J=8.2 Hz), 4.50 (2H, s), 2.94 (2H, t, J=7.4 Hz), 1.74-1.71 (2H, m), 1.38-1.26 (17H, m), 0.88 (3H, t, J=6.9 Hz). ¹³C NMR (126 MHz, CDCl₃): δ 199.9, 142.5, 136.9, 129.2, 128.6, 38.7, 32.2, 31.9, 29.7, 29.6, 29.50, 29.47, 29.36, 29.33, 24.34, 22.69, 14.12. MS (ESI+): Exact Mass Calcd for [C₁₉H₃₀BrO]+ (M+H+)=353.2 and 355.2. Found 353.2 and 355.2.

### Synthesis and Characterization of Lauroyl MBEH (Compound 3).

This synthesis was adapted from a published procedure for the synthesis of 1-(5-(benzyloxy)-2-hydroxyphenyl)ethanone. [Mays J R, Hill S A, Moyers J T, Blagg B S. The synthesis and evaluation of flavone and isoflavone chimeras of novobiocin and derrubone. Bioorg. Med. Chem. 2010; 18(1):249-66.] Potassium carbonate (534.8 mg, 3.87 mmol, 1 equiv) and compound 2 (1.36 g, 3.85 mmol, 1 equiv) were added in sequence to a solution of hydroquinone (424.7 mg, 3.86 mmol, 1 equiv) in acetone (10 mL) in a 100 mL round-bottom flask equipped with a magnetic stir bar. A water-cooled reflux condenser was attached to the flask and the reaction mixture was then heated to reflux for 48 h. After cooling to t, the reaction mixture was combined with saturated aqueous NaHCO₃ solution (10 mL) and the resulting mixture was extracted with EtOAc (3×20 mL). The combined organic layers were washed with saturated aqueous NaCl solution (100 mL), dried over MgSO₄, filtered, and concentrated to dryness. The residue was purified via flash column chromatography over silica gel (5:1 v/v hexanes/EtOAc) to give lauroyl MBEH (3) as a white amorphous solid (450 mg, 31%).

The structure and composition of compound 3 was confirmed by NMR spectroscopy and mass spectrometry. ¹H NMR (CDCl₃, 500 MHz): δ 8.10-7.96 (2H, m), 7.51-7.45 (2H, m), 6.90-6.84 (2H, m), 6.79-6.76 (2H, m), 5.14-5.02 (2H, m), 2.97-2.94 (2H, m), 1.74-1.72 (2H, m), 1.26 (16H, s, br), 0.89-0.86 (3H, m). ¹³C NMR (126 MHz, CDCl₃): δ 200.3, 152.7, 149.9, 142.4, 136.6, 128.4, 127.2, 116.1, 116.0, 70.1, 38.7, 31.9, 29.6, 29.6, 29.5, 29.4, 29.3, 24.4, 22.7, 14.1. MS (ESI−): Exact Mass Calcd for [C₂₅H₃₃O₃]⁻ (M−H)⁻=381.2. Found 380.99. Exact Mass Calcd for dimer [C₅₀H₆₇O₆]⁻ (2M−H)⁻=763.49. Found 763.56.

### Preparation and Characterization of DOPC Liposomes.

DOPC-based liposomes can be synthesized in high yields (>90%) at room temperature (rt) due to the very low T. of DOPC lipid (−17° C.). The synthesized liposomes can be kept in a refrigerator and used for biological studies.

The DOPC lipid (31.5 mg, 42 μmol) was completely dissolved in HPLC-grade chloroform (1 mL) in a 50 mL round-bottom flask and then evaporated under reduced pressure by a rotary evaporator. The resulting lipid film was thoroughly dried on a Schlenk line for 12 h more before being combined with 20 mM sterile HBS (20 mM HEPES and 150 mM NaCl; corrected to pH 7.4 using HCl_{aq} (1 and 5 M) after the addition of NaHEPES salt to sterile saline; 4.0 mL). [Hong B J, Iscen A, Chipre A J, Li M M, Lee O-S, Leonard J N, Schatz G C, Nguyen S T. Highly stable, ultrasmall polymer-grafted nanobins (usPGNs) with stimuli-responsive capability. J. Phys. Chem. Let. 2018; 9(5):1133-1139] Using an Avanti Mini-Extruder, this turbid suspension was extruded at least 40 times through a 0.1-micron polycarbonate membrane (Avanti Polar Lipids 610005) at rt to give a clear solution of liposomes. [Onyango J O, Chung M S, Eng C-H, Klees L M, Langenbacher R, Yao L, An M. Noncanonical Amino Acids to Improve the pH Response of pHLIP Insertion at Tumor Acidity. Angew. Chem. Inter. Ed. 2015; 54(12):3658-3663] The solution (˜4 mL) contains liposomes with a mean hydrodynamic diameter of 124.4 nm (PDI=0.101) as determined by DLS measurements. [Lee S-M, Chen H, Dettmer C M, O'Halloran T V, Nguyen S T. Polymer-caged liposomes: a pH-responsive delivery system with high stability. J. Am. Chem. Soc 2007; 129(49):15096-15097] This solution (˜4 mL) was then passed through a Sephadex G-50 column (fraction range: 1,500-30,000 Da, 2 cm×7 cm) that was wet-packed and pre-equilibrated with 10-mM sterile PIPES solution (corrected to pH 7.4 using NaOH_{aq} (1 and 5 M) after the addition of PIPES to sterile saline, 150 mM NaCl) to remove any left-over free DOPC lipid. The eluant was the same 10 mM PIPES solution. The liposome-containing elution was confirmed with a laser pointer and collected as a single fraction.

The phospholipid concentration in the liposome-containing fraction was measured using ICP-OES. Briefly, to prepare a sample for ICP-OES measurement, an aliquot (20 μL) of DOPC liposomes was diluted in nitric acid (c=0.5 mol L⁻¹) to a total volume of 5 mL. This mixture was vortexed (1 min) thoroughly before being left to equilibrate for a minimum of 3 h before being subjected to ICP-OES analysis. The concentration of lipids in the purified liposome sample was determined to be 5.43 mM. [Fenzl C, Hirsch T, Baeumner A J. Liposomes with high refractive index encapsulants as tunable signal amplification tools in surface plasmon resonance spectroscopy. Anal. Chem. 2015; 87(21):11157-11163]

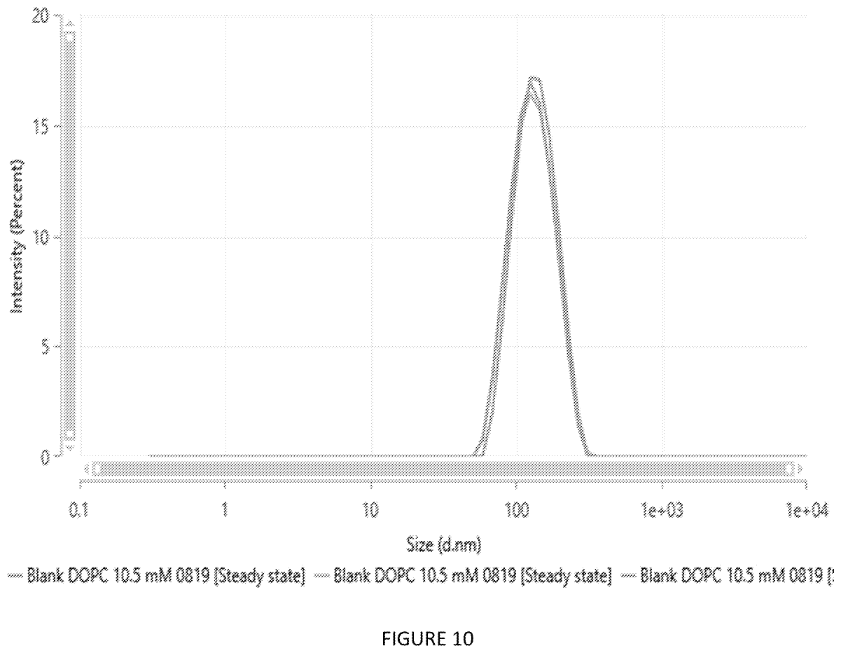

FIG. 10 shows dynamic light scattering (DLS) traces of the as-synthesized DOPC-liposomes.

### Preparation and Characterization of Lauroyl-MBEH-Loaded DOPC Liposomes.

The lauroyl MBEH (4.5 mg, 12 μmol) and DOPC lipid (94.5 mg, 120 μmol) was completely dissolved in a 1:1 v/v mixture of HPLC-grade chloroform and EtOAc (2 mL) in a 50 mL round-bottom flask and then evaporated under reduced pressure by a rotary evaporator. The resulting lipid film was thoroughly dried on a Schlenk line for 12 h more before being combined with 20 mM sterile HBS (20 mM HEPES and 150 mM NaCl; corrected to pH 7.4 using HCl_{aq} (1 and 5 M) after the addition of NaHEPES salt to sterile saline; 8.0 mL). Lipid hydration was accomplished by vigorous vortexing on an Vortex Mixer (American Scientific Products, Charlotte, NC) for 5 min. [Hong B J, Iscen A, Chipre A J, Li M M, Lee O-S, Leonard J N, Schatz G C, Nguyen S T. Highly stable, ultrasmall polymer-grafted nanobins (usPGNs) with stimuli-responsive capability. J. Phys. Chem. Lett. 2018; 9(5):1133-1139] After being subjected to 10 freeze-thaw-under-sonication (˜10 min.) cycles, the resulting dispersion of lauroyl-MBEH-loaded liposomes was extruded through hydrophilic polycarbonate track-etched membranes with a stepping down extrusion method. Briefly, on a mini-extruder block the dispersion was extruded ten times through a two-stack membranes (800-nm pore-size), ten times through a stack of one 800-nm and one 400-nm membranes, ten times through a two stack 400-nm membranes, ten times through a stack of one 400-nm and one 200-nm membrane, ten times through a two-stack 200 nm membranes, ten times through a stack of one 200-nm and one 100-nm membrane, and then ten times through a two-stack 100-nm membranes. At the end, the initial vial and syringes were washed with an additional aliquot of HBS buffer (300 μL) and this wash was also extruded ten times through a two-stack 100-nm membranes. The combined solution (˜8 mL) was passed the incubated solution through a Sephadex G-50 column (fraction range: 1,500-30,000 Da, 2 cm×7 cm) that was wet-packed and pre-equilibrated with 10-mM sterile PIPES solution (corrected to pH 7.4 pH 7.4 using NaOH_{aq} (1 and 5 M) after the addition of PIPES to sterile saline, 150 mM NaCl) to remove any free unloaded lauroyl MBEH and/or non-liposomal lipid rafts. The eluant was also 10-mM PIPES solution and the fractions was collected every 5 drops. The drug-loaded-liposome-containing fractions can be easily recognized by their turbidity and the most concentrated ones were combined together (˜4 mL). The hydrodynamic diameter (DH) of the lauroyl-MBEH-loaded liposomes is 81.9 nm (PDI=0.0034). The final concentration of lipids was 15.016 mM as determined by phosphorous ICP-OES. [Lee S-M, Chen H, Dettmer C M, O'Halloran T V, Nguyen S T. Polymer-caged liposomes: a pH-responsive delivery system with high stability. J. Am. Chem. Soc 2007; 129(49):15096-15097]

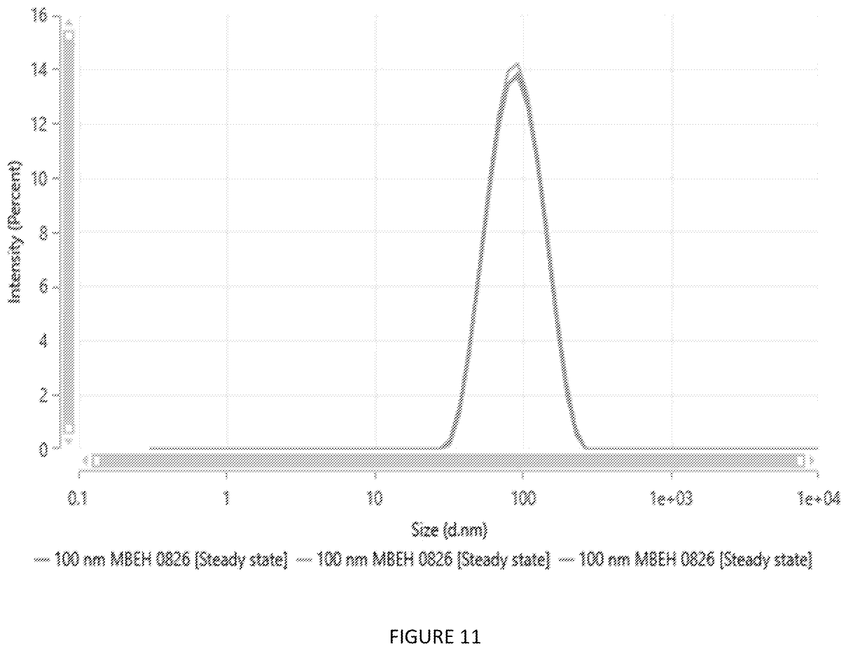

FIG. 11 shows DLS traces of the lauroyl-MBEH-loaded DOPC liposomes.

Preparation and Characterization of Lauroyl-MBEH-Loaded Liposomes Labeled with Rhod-PE.

This dye-labeled liposome was made following the above preparation procedure of the drug-loaded liposomes, except that the Rhod-PE was added. Briefly, Rhod-PE (1 mg, 0.8 μmol) was added to the solution of lauroyl MBEH (3 mg, 8 μmol) and DOPC lipid (63 mg, 80 μmol) in chloroform and EtOAc. Then the dye-labeled drug-loaded liposomes were prepared in 20 mM sterile HBS buffer. After purification, the solution contains liposomes with a mean hydrodynamic diameter of 90.63 nm (PDI=0.049) as determined by DLS (dynamic light scattering) measurements. The final concentration of lipids was 5.99 mM as determined by phosphorous ICP-OES (inductively coupled plasma optical emission spectroscopy). [Lee S-M, Chen H, Dettmer C M, O'Halloran T V, Nguyen S T. Polymer-caged liposomes: a pH-responsive delivery system with high stability. J. Am. Chem. Soc 2007; 129(49):15096-15097]

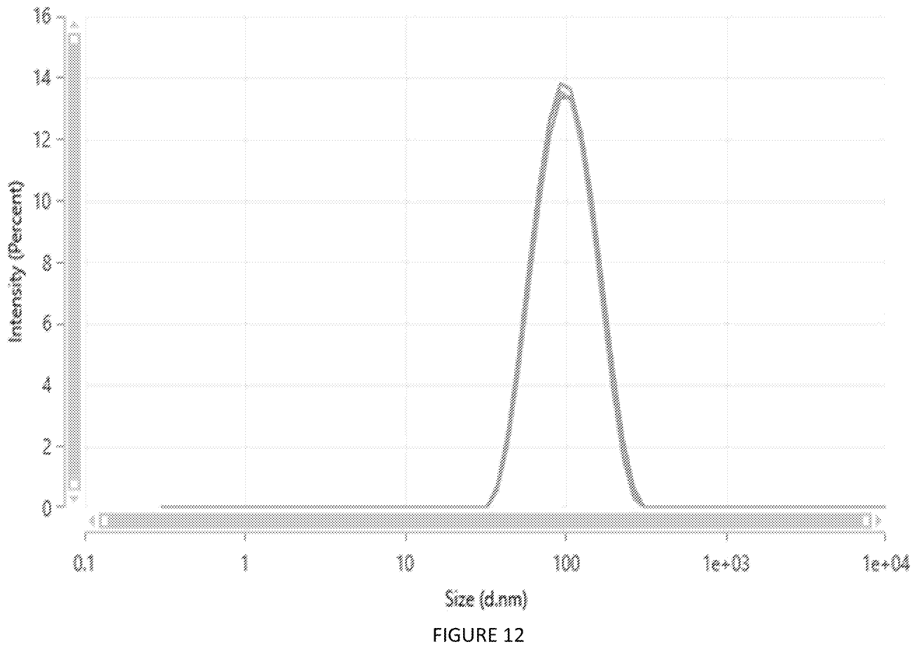

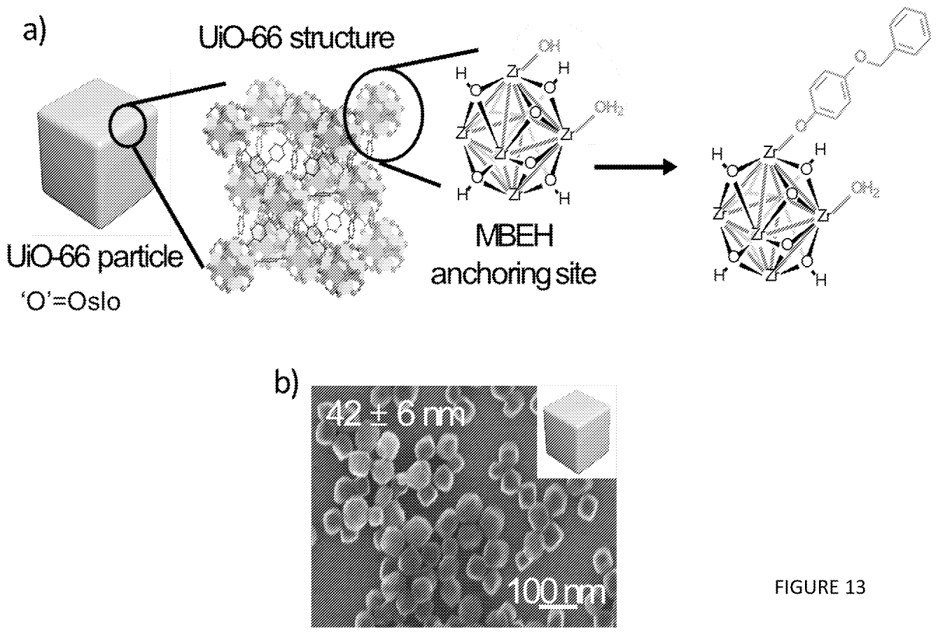

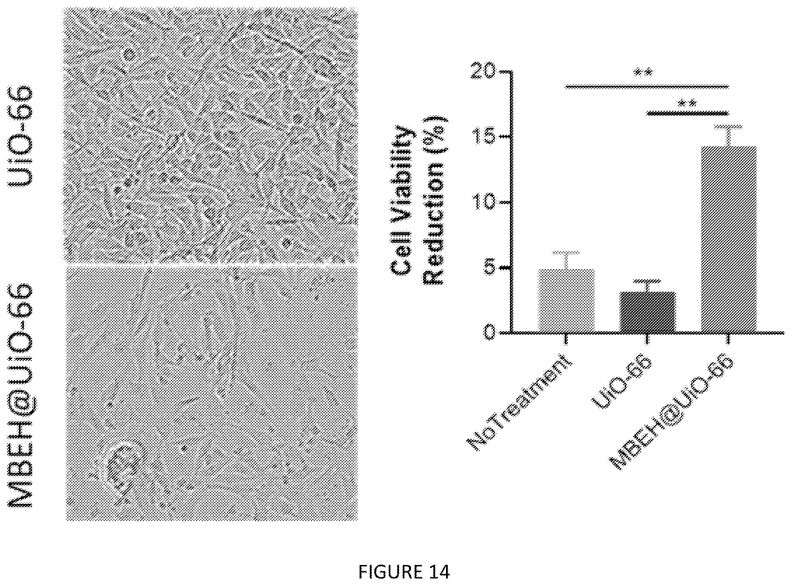

FIG. 12 shows DLS traces of the Rhod-PE-labeled, lauroyl-MBEH-loaded liposomes.

### Exemplary Methods for Preparing Monobenzone Conjugates

Monobenzone conjugates may be prepared by the addition of hydroquinone to a substituted (bromomethyl)phenyl as shown in Scheme 2 where R may be an alkyl, aryl, —X-alkyl, or —X-aryl where X is a linking group such as —C(═O)—, —C(═O)O—, —C(═O)N(H)—, —O—, —N(H)—, —N(alkyl)-, or a protected group thereof.

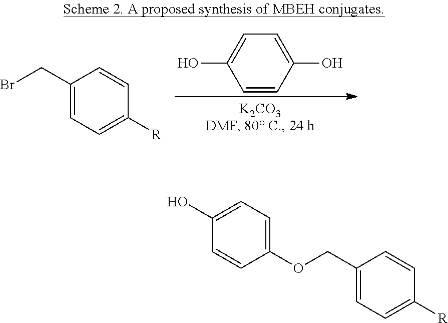

Proposed synthetic routes to several MBEH conjugates are shown in Schemes 3-6.

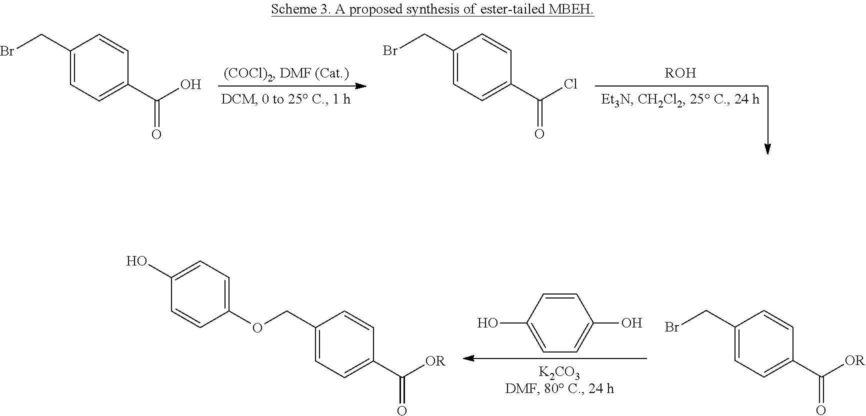

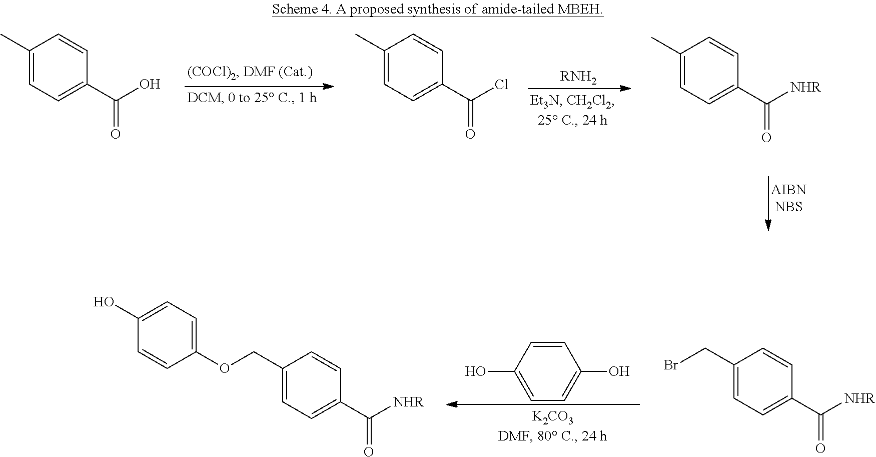

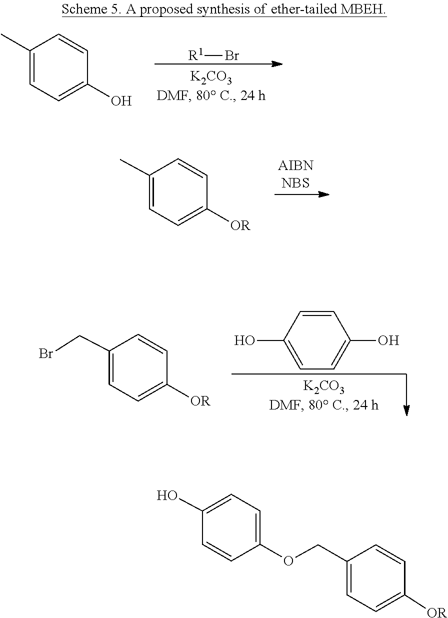

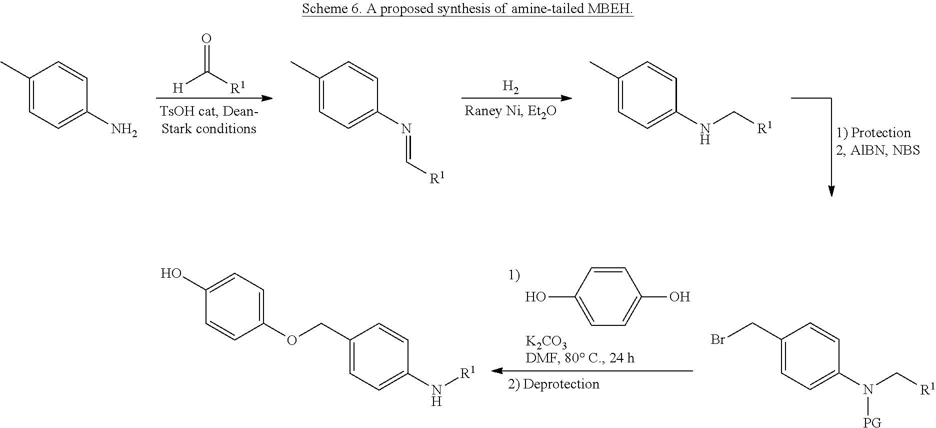

### REFERENCES

1. Garbe C, Peris K, Hauschild A, Saiag P, Middleton M, Bastholt L, Grob J-J, Malvehy J, Newton-Bishop J, Stratigos A J. Diagnosis and treatment of melanoma. European consensus-based interdisciplinary guideline—Update 2016. Eur. J. Cancer 2016; 63:201-17.
2. Albertini M R. The age of enlightenment in melanoma immunotherapy. J. Immunother. Cancer 2018; 6(1):80-3.
3. Manini P, Napolitano A, Westerhof W, Riley P A, d'Ischia M. A reactive ortho-quinone generated by tyrosinase-catalyzed oxidation of the skin depigmenting agent monobenzone: self-coupling and thiol-conjugation reactions and possible implications for melanocyte toxicity. Chem. Res. Toxicol. 2009; 22:1398-405.
4. Hariharan V, Klarquist J, Reust M J, Koshoffer A, McKee M D, Boissy R E, Le Poole I C. Monobenzyl ether of hydroquinone and 4-tertiary butyl phenol activate markedly different physiological responses in melanocytes: relevance to skin depigmentation. J. Invest. Dermatol. 2010; 130(1):211-20. Epub 2009/08/07.
5. Hariharan V, Toole T, Klarquist J, Longley J B, Mosenson J, Le Poole I C. Topical application of bleaching phenols; in vivo studies and mechanism of action relevant to melanoma treatment. Melanoma Res. 2011; 21(2):115-26.
6. Teulings H-E, Tjin E P, Willemsen K J, van der Kleij S, Meulen St, Kemp E H, Krebbers G, van Noesel C J, Franken C L, Drijfhout J W. Anti-Melanoma immunity and local regression of cutaneous metastases in melanoma patients treated with monobenzone and imiquimod; a phase 2 a trial. Oncoimmunology 2018; 7(4):e1419113-e27.
7. de Souza Salgado Y C, Ferreira M B, da Luz J Z, Neto F F, de Oliveira Ribeiro C A. Tribromophenol affects the metabolism, proliferation, migration and multidrug resistance transporters activity of murine melanoma cells BI6F1. Toxicol. in Vitro 2018; 50:40-6.
8. Farokhzad O C, Langer R. Impact of nanotechnology on drug delivery. ACS Nano 2009; 3(1):16-20.
9. Chen D, Yang D, Dougherty C A, Lu W, Wu H, He X, Cai T, Van Dort M E, Ross B D, Hong H. In vivo targeting and positron emission tomography imaging of tumor with intrinsically radioactive metal-organic frameworks nanomaterials. ACS Nano 2017; 11(4):4315-27.
10. Zhang J, Chang D, Yang Y, Zhang X, Tao W, Jiang L, Liang X, Tsai H, Huang L, Mei L. Systematic investigation on the intracellular trafficking network of polymeric nanoparticles. Nanoscale 2017; 9(9):3269-82.
11. Le Poole I C, van den Wijngaard R M J G J, Westerhof W, Verkruisen R P, Dutrieux R P, Dingemans K P, Das P K. Phagocytosis by Normal Human Melanocytes in Vitro. Exp. Cell Res. 1993; 205(2):388-95.
12. Hong B J, Iscen A, Chipre A J, Li M M, Lee O-S, Leonard J N, Schatz G C, Nguyen S T. Highly stable, ultrasmall polymer-grafted nanobins (usPGNs) with stimuli-responsive capability. J. Phys. Chem. Lett. 2018; 9(5):1133-9.
13. Molnar D, Linders J, Mayer C, Schubert R. Insertion stability of poly(ethylene glycol)-cholesteryl-based lipid anchors in liposome membranes. Eur. J. Pharm. Biopharm. 2016; 103:51-61.
14. Ramos R I, Bustos M A, Wu J, Jones P, Chang S C, Kiyohara E, Tran K, Zhang X, Stern S L, Izraely S, Sagi-Assif O, Witz I P, Davies M A, Mills G B, Kelly D F, Irie R F, Hoon D S B. Upregulation of cell surface GD3 ganglioside phenotype is associated with human melanoma brain metastasis. Mol. Oncol. 2020; 14(8):1760-78. Epub 2020/05/03.
15. Hong B J, Swindell E P, MacRenaris K W, Hankins P L, Chipre A J, Mastarone D J, Ahn R W, Meade T J, O'Halloran T V, Nguyen S T. pH-responsive theranostic polymer-caged nanobins: Enhanced cytotoxicity and T1 MRI contrast by HER-2 targeting. Part. Part. Syst. Charact. 2013; 30(9):770-4.
16. Kammeyer A, Willemsen K J, Ouwerkerk W, Bakker W J, Ratsma D. Pronk S D, Smit N P M, Luiten R M. Mechanism of action of 4-substituted phenols to induce vitiligo and antimelanoma immunity. Pigment Cell & Melanoma Research 2019; 32(4):540-52.
17. Yu P, Hu J, Zhou T-Y, Wang P, Xu Y-H. Synthesis, insecticidal evaluation of novel 1,3,4-thiadiazole chrysanthemamide derivatives formed by an edci/hobt condensation. J. Chem. Res. 2011; 35(12):703-6.
18. Yang Y, Yu Y, Li X, Li J, Wu Y, Yu J, Ge J, Huang Z, Jiang L, Rao Y. Target elucidation by cocrystal structures of NADH-ubiquinone oxidoreductase of Plasmodium falciparum (PJNDH2) with small molecule to eliminate drug-resistant malaria. J. Med. Chem. 2017; 60(5):1994-2005.
19. Mays J R, Hill S A, Moyers J T, Blagg B S. The synthesis and evaluation of flavone and isoflavone chimeras of novobiocin and derrubone. Bioorg. Med. Chem. 2010; 18(1):249-66.
20. Hong B J, Iscen A, Chipre A J, Li M M, Lee O-S, Leonard J N, Schatz G C, Nguyen S T. Highly stable, ultrasmall polymer-grafted nanobins (usPGNs) with stimuli-responsive capability. J. Phys. Chem. Lett. 2018; 9(5):1133-9.
21. Onyango J O, Chung M S, Eng C-H, Klees L M, Langenbacher R, Yao L, An M. Noncanonical Amino Acids to Improve the pH Response of pHLIP Insertion at Tumor Acidity. Angew. Chem. Inter. Ed. 2015; 54(12):3658-63.
22. Lee S-M, Chen H, Dettmer C M, O'Halloran T V, Nguyen S T. Polymer-caged liposomes: a pH-responsive delivery system with high stability. J. Am. Chem. Soc 2007; 129(49):15096-7.
23. Fenzl C, Hirsch T, Baeumner A J. Liposomes with high refractive index encapsulants as tunable signal amplification tools in surface plasmon resonance spectroscopy. Anal. Chem. 2015; 87(21):11157-63.
24. Zhou Y, Wu J, Farajtabar A, Wang J, Zhao H. Solubility of monobenzone in aqueous co-solvent mixtures of several alcohols: Determination, modelling and thermodynamic aspects analysis. J. Chem. Thermo. 2020; 142:106023-32.
25. Cheng Z, Elias D R, Kamat N P, Johnston E D, Poloukhtine A, Popik V, Hammer D A, Tsourkas A. Improved tumor targeting of polymer-based nanovesicles using polymer-lipid blends. Bioconjug Chem 2011; 22(10):2021-9. Epub 2011/09/20.

1. Nanoparticulate monobenzone comprising a nanostructured carrier and monobenzone or a conjugate thereof embedded within the nanostructured carrier.

2. The nanoparticulate monobenzone of claim 1, wherein the loading of the monobenzone or the monobenzone conjugate is in the 5-25 mol % range.

3. The nanoparticulate monobenzone of claim 1, wherein the particulate monobenzone has a hydrodynamic diameter (DH) from 30 to 120 nm.

4. The nanoparticulate monobenzone of claim 3, wherein the loading of the monobenzone or the monobenzone conjugate is in the 5-25 mol % range.

5. The nanoparticulate monobenzone of claim 1 comprising a liposomal carrier and the monobenzone conjugate, wherein the monobenzone conjugate comprises an alkyl, aryl, —X-alkyl, or —X-aryl moiety covalently bound to monobenzone and X is selected from —C(═O)—, —C(═O)O—, —C(═O)N(H)—, —O—, —N(H)—, or —N(alkyl)-.

6. The nanoparticulate monobenzone of claim 5, wherein the monobenzone conjugate comprises a compound of formula 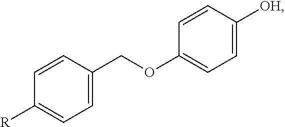
wherein R is —X-alkyl and the alkyl is an unbranched or branched, saturated or unsaturated C₄-C₂₈ alkyl or —X-aryl and the aryl is a C₆-C₁₀ aryl.

7. The nanoparticulate monobenzone of claim 6, wherein the monobenzone conjugate is

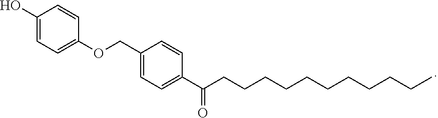 [SMILES: CCCCCCCCCCCC(=O)c1ccc(COc2ccc(O)cc2)cc1]

8. The nanoparticulate monobenzone of claim 5, wherein the monobenzone conjugate comprises a compound of formula 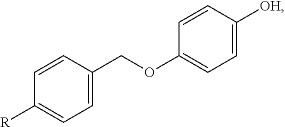
wherein
R is -alkyl and the alkyl is an unbranched or branched, saturated or unsaturated C₄-C₂₈ alkyl or -aryl and the aryl is a C₆-C₁₀ aryl.

9. The nanoparticulate monobenzone of claim 8, wherein the monobenzone conjugate is

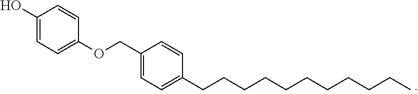 [SMILES: CCCCCCCCCCCc1ccc(COc2ccc(O)cc2)cc1]

10. The nanoparticulate monobenzone of claim 1 comprising a liposomal carrier and monobenzone.

11. The nanoparticulate monobenzone of claim 5, wherein the liposomal carrier further comprises a detectable label, a stabilizing agent, a targeting agent, linking agent, or any combination thereof.

12. The nanoparticulate monobenzone of claim 1 comprising a metal-organic framework (MOF) carrier.

13. The nanoparticulate monobenzone of claim 12, wherein the MOF carrier is UiO-66.

14. The nanoparticulate monobenzone of claim 12 comprising monobenzone.

15. A pharmaceutical composition comprising the nanoparticulate monobenzone according to claim 1 and a pharmaceutically acceptable carrier, diluent, or excipient.

16. A method of the treatment of a subject comprising administering the nanoparticulate monobenzone according to claim 1 wherein the subject is in need of a treatment for melanoma.

17. The method of claim 16, wherein the nanoparticulate monobenzone is systemically administered.

18-27. (canceled)

28. A method for the preparation of nanoparticulate monobenzone, the method comprising:
preparing a composition comprising a lipid film and monobenzone or a conjugate thereof and
forming a liposomal carrier comprising and monobenzone or the conjugate thereof embedded within the liposomal carrier.

29. The method of claim 28, wherein forming the liposomal carrier comprises:
hydrating the lipid film;
forming a dispersion by subjecting the hydrated lipid film to one or more freeze-thaw cycles under sonication; and
extruding the dispersion through one or more membranes.

30. The method of claim 29, wherein the dispersion is extruded repeatedly through a two-stack membrane.

31-33. (canceled)
